SEQUENCE LISTING (10 sequences)

SEQ ID NO: 1 - protein, 5 aa, Artificial Sequence
  ETTES peptide
ETTES

SEQ ID NO: 2 - protein, 5 aa, Artificial Sequence
  KTTKS peptide
  MISC_FEATURE (1)..(1): C16
KTTKS

SEQ ID NO: 3 - protein, 4 aa, Artificial Sequence
  cell adhesion moiety
RGDS

SEQ ID NO: 4 - protein, 4 aa, Artificial Sequence
  ETTE peptide
  MISC_FEATURE (1)..(1): C16
ETTE

SEQ ID NO: 5 - protein, 4 aa, Artificial Sequence
  ETTES Fragment
TTES

SEQ ID NO: 6 - protein, 5 aa, Artificial Sequence
  ETTES Variant
ETETS

SEQ ID NO: 7 - protein, 5 aa, Artificial Sequence
  ETTES Variant
TTEES

SEQ ID NO: 8 - protein, 5 aa, Artificial Sequence
  ETTES Variant
SEETT

SEQ ID NO: 9 - protein, 5 aa, Artificial Sequence
  ETTES Variant
SETET

SEQ ID NO: 10 - protein, 5 aa, Artificial Sequence
  ETTES Variant
TESTE

(19) United States
(12) Patent Application Publication
(10) Pub. No.: US 2023/0002448 A1
(43) Pub. Date: Jan. 5, 2023

(54) COLLAGEN PRODUCTION

(71) Applicant: 3D Bio-tissues Limited, Newcastle Upon Tyne (GB)

(72) Inventors: Martina MIOTTO, Newcastle Upon Tyne (GB); Che John CONNON, Newcastle Upon Tyne (GB); Ricardo GOUVEIA, Newcastle Upon Tyne (GB)

(21) Appl. No.: 17/782,948

(22) PCT Filed: Dec. 11, 2020
(86) PCT No.: PCT/GB2020/053190

§ 371 Date: Jun. 6, 2022

(30) Foreign Application Priority Data
Dec. 11, 2019 (GB) 1918167.6

(51) Int. Cl. C07K 7/06; C12N 5/079; A61K 8/64; A61Q 19/08; A61Q 19/02; A61P 17/00

(52) U.S. Cl. CPC C07K 7/06; C12N 5/0621; A61K 8/64; A61Q 19/08; A61Q 19/02; A61P 17/00; A61K 38/00

(57) ABSTRACT

The present invention provides a method for increasing collagen production in a cell and a method for inhibiting cell migration. Further, the present invention provides a pharmaceutical composition comprising lipopeptides, wherein the lipopeptides substantially consist of ETTES lipopeptides, and uses of said pharmaceutical composition. The invention also provides a supramolecular structure comprising lipopeptides, wherein the lipopeptides substantially consist of ETTES lipopeptides, as well as a method for producing said supramolecular structure. The supramolecular structure of the invention may be used in the method for increasing collagen production and/or method for inhibiting cell migration.

The present invention provides a method for increasing collagen production in a cell and a method for inhibiting cell migration. Further, the present invention provides a pharmaceutical composition comprising lipopeptides, wherein the lipopeptides substantially consist of ETTES lipopeptides, and uses of said pharmaceutical composition. The invention also provides a supramolecular structure comprising lipopeptides, wherein the lipopeptides substantially consist of ETTES lipopeptides, as well as a method for producing said supramolecular structure. The supramolecular structure of the invention may be used in the method for increasing collagen production and/or method for inhibiting cell migration.

### BACKGROUND

The regeneration of connective tissues such as cornea, muscle, skin, cartilage, and bone, depends both on the re-deposition of healthy extracellular matrix (ECM), which includes collagen, as well as on the maintenance of resident cells with tissue-specific phenotypes. Some peptide amphiphile (PA) molecules, also referred to as lipopeptides, have been shown as potential candidates for use in a clinical procedure that allows tissue-specific cells to repair and regenerate the ECM whilst preventing the formation of scar tissue.

PAs are synthetic materials used for their ability to self-assemble in aqueous media at physiological pH into highly ordered nanostructures. Some of these nanostructures have been shown to have unique effects on cell viability and/or protein expression. For instance, 016-KTTKS lipopeptides, used under the trade name of Matrixyl, have been shown to stimulate collagen production in vitro in corneal and skin fibroblasts (Jones et al., 2013).

The present invention aims to provide alternative and/or improved PAs with a variety of potential applications, for example in corneal tissue regeneration after injury, in vitro tissue biofabrication, or in skin care.

### SUMMARY OF THE INVENTION

The present invention is based on the inventors' surprising finding that a C₁₆-ETTES lipopeptide has a number of unexpected uses and advantages.

The C₁₆-ETTES lipopeptide has been previously considered bio-inert and has been used as a non-bioactive diluent molecule to assist with cell attachment by co-assembling lipopeptides with bioactive moieties (such as cell adhesion moieties for example RGD or RGDS).

As a diluent, the C₁₆-ETTES lipopeptide is able to vary the density of the bioactive moieties within the supramolecular structure formed by assembled lipopeptides. The functional amino acid sequence of the C₁₆-ETTES lipopeptide was initially rationally designed as a non-bioactive diluent molecule able to optimise the distance between other neighbouring lipopeptide molecules (Castelletto et al., 2013). It was shown that by using the C₁₆-ETTES lipopeptide, the distance between neighbouring lipopeptides with cell adhesion moieties such as RGD or RGDS could be altered which allowed improved cell attachment to the cell adhesion moieties. A lipopeptide mixture comprising the C₁₆-ETTES lipopeptide was subsequently used in a RGDS:ETTES coating for 2D human corneal stromal fibroblast (hCSF) attachment and growth. The coating has been described to function not only as a support for hCSF adhesion but also as an effector in tuning the cell phenotype and preventing cell death in serum-free conditions. Specifically, results have indicated that the RGDS:ETTES coating not only increased hCSF adhesion and proliferation, but also enhanced the molecular and morphological phenotypes characteristic of hCSFs grown in serum-free conditions for long periods in culture. In this type of coating the C₁₆-ETTES lipopeptide was used only a diluent molecule, but was not shown to have any bioactive function.

Surprisingly, the present inventors have found that culturing cells in the presence of the 016-ETTES lipopeptide increases the amount of extracellular matrix such as collagen produced by the cells. This beneficial effect was observed by the inventors in cells such as stromal cells (for example corneal stromal cells), adipose-derived mesenchymal stem cells (hASCs), and myoblasts. Without wishing to be bound to a specific hypothesis, the inventors believe that the C₁₆-ETTES lipopeptides are able to increase extracellular matrix (for example collagen) production by providing a nucleation point for extracellular collagen fibrilization and/or by acting as a ligand to a cell receptor (for example interphotoreceptor matrix proteoglycan 1 receptor (IMPG1)). Additionally, the inventors have also found that culturing of cells in the presence of the C₁₆-ETTES lipopeptide may inhibit cell migration. The inventors believe that there may be a direct relationship between increasing collagen production and decreasing cell migration.

Furthermore, the inventors have found that C₁₆-ETTES lipopeptides have this beneficial effect regardless of whether the lipopeptides were assembled into a supramolecular structure in water or solvents having an ionic strength greater than water, such as cell culture medium. Self-assembly in the solvent, however, results in the lipopeptides forming a novel supramolecular structure with a unique, globular topology. A supramolecular structure with such a topology may be referred to herein as a fibrillar supramolecular structure.

These unexpected findings give rise to the various aspects of the invention described herein.

In one aspect, the present invention provides a method for increasing collagen production in a cell, the method comprising the step of contacting the cell with lipopeptides, wherein the lipopeptides substantially consist of ETTES lipopeptides.

In one aspect, the present invention provides a method for inhibiting cell migration, the method comprising the step of contacting the cell with lipopeptides, wherein the lipopeptides substantially consist of ETTES lipopeptides.

In one aspect, the present invention provides a pharmaceutical composition comprising lipopeptides, wherein the lipopeptides substantially consist of ETTES lipopeptides.

In one aspect, the present invention provides a pharmaceutical composition for use in therapy, wherein the composition comprises lipopeptides, wherein the lipopeptides substantially consist of ETTES lipopeptides.

In one aspect, the present invention provides a pharmaceutical composition for use in the treatment of a collagen deficiency disease, use in enhancing wound healing, and/or for use in the treatment of cancer, wherein the composition comprises lipopeptides, wherein the lipopeptides substantially consist of ETTES lipopeptides.

In one aspect, the present invention provides a method of treating a collagen deficiency disease, wound healing, or treating cancer in a subject, the method comprising the step of administering to the subject a therapeutically effective amount of a pharmaceutical composition comprising lipopeptides, wherein the lipopeptides substantially consist of ETTES lipopeptides.

In one aspect, the present invention provides use of a pharmaceutical composition comprising lipopeptides, wherein the lipopeptides substantially consist of ETTES lipopeptides.

In one aspect, the present invention provides a fibrillar supramolecular structure comprising lipopeptides, wherein the lipopeptides substantially consist of ETTES lipopeptides.

In one aspect, the present invention provides a method of producing a fibrillar supramolecular structure comprising lipopeptides, wherein the lipopeptides substantially consist of ETTES lipopeptides, the method comprising dissolving the lipopeptides in a solvent having an ionic strength that is greater than the ionic strength of distilled water to produce the supramolecular structure.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural and vice versa unless the context otherwise requires. Where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. Compatibility of features will be recognised by those skilled in the art.

As mentioned herein, the inventors have found that there may be a correlation between inhibiting cell migration and increasing collagen production in the cell. As such, it will be appreciated that each of the embodiments or examples disclosed herein in the context of a method for increasing collagen production are also applicable to the method for inhibiting cell migration.

Various aspects and embodiments of the invention are described in further detail below.

### BRIEF DESCRIPTION OF THE FIGURES

Embodiments of the invention are further described hereinafter with reference to the accompanying drawings, in which:

FIG. 1 shows the ultrastructure of the C₁₆-ETTES PA self-assembled in serum-free medium (SFM) (A) and deionised water (B). AFM false colour scale: 50 nm. Scale bars: 5 μm (left) and 1 μm (right panels).

FIG. 2 shows the effect of different concentrations of C₁₆-ETTES PA on hCSF proliferation at day 3 and 7 of culture having previously been self-assembled in (A) SFM and (B) water. Quantification was performed using Alamar Blue assay. (C) Images of micrographs showing hCSF viability after 7 days in culture with different concentrations of C₁₆-ETTES PA (scale bars: 250 μm). Mean±S.D., n=3 for all experiments; *** and **** referred to statistically significant differences compared to the control and corresponded to p<0.001 and p<0.0001, respectively.

FIG. 3 shows the effect of different concentrations of C₁₆-ETTES PA on bulk collagen deposition by hCSFs after 7 days in culture (A), as well as the collagen produced per cell (B), using the Sirius-red/picric acid assay. Mean±S.D., n=3 for all experiments; * referred to statistically significant differences compared to the control and corresponded to p<0.05. (C) Micrographs illustrating the deposited collagen stained with Sirius-red/picric acid after 7 days of culture in different conditions (scale bars: 200 μm).

FIG. 4 shows the effect of SFM-solubilised C₁₆-ETTES lipopeptide and Matrixyl PAs on hCSF migration. The graph (A) reports the % of wound closure of the scratch assay performed on hCSFs after being cultured for 2 days either in absence (CTR SFM) or in presence of ETTES and Matrixyl PAs at 50 μM and (B) shows the corresponding total amount of deposited collagen after the scratch using the Sirius-red/picric acid assay. Mean±S.D., n=3 for all experiments; * and **** referred to statistically significant differences compared to the control (CTR SFM) and corresponded to p<0.05 and p<0.0001, respectively. Scale bars: 250 μm.

FIG. 5 shows the effect of C₁₆-ETTES lipopeptide on collagen deposition by hASCs. (A) Shows the total amount of collagen deposited by hASCs after 7 days in culture with ETTES PA at 50 and 500 μM, assessed using the Sirius-red/picric acid assay. Mean±S.D., n=3 for all experiments; * referred to statistically significant differences compared to the control and corresponded to p<0.05. (B) are images of micrographs showing the deposited collagen stained with Sirius-red/picric acid after 7 days of culture in different conditions (scale bars: 150 μm).

FIG. 6 shows graphs illustrating the effect of different concentrations of C₁₆-ETTES PA on myoblast proliferation at day 3 and 7 of culture when prepared with SFM or water (A). Quantification was performed using Alamar Blue assay. (B) Is a graph illustrating the total amount of collagen deposited by myoblasts after 7 days in culture with ETTES PA at 25 and 50 μM, assessed using the Sirius-red/picric acid assay. Mean±S.D., n=3 for all experiments; * and **** referred to statistically significant differences compared to the control and corresponded to p<0.05 and 0.0001, respectively.

FIG. 7 shows graphs illustrating corneal stromal cell collagen deposition (by Sirius Red assay) at various molar ratios of RGDS:ETTES at 500 μM and 50 μM relative to control in SFM (0:0 molar ratio) after 7 days in culture. (Average±S.D.; * corresponded to p<0.05). It can be seen that greater ratio of ETTES to RGDS results in increased collagen deposition.

FIG. 8 shows the effect of C₁₆-ETTES PAs with different lipid portion chain lengths on hCSF proliferation at day 3 and 7 of culture. Specifically, the C₈-ETTES and C₂₀-ETTES PAs were self-assembled in (A) SFM and (B) water, and their effect compared with that of C₁₆-ETTES. Quantification was performed using Alamar Blue assay. Mean±S.D., n=3 for all experiments; n.s., no statistically-significant differences between variants and the C₁₆-ETTES control.

FIG. 9 shows the effect of C₁₆-ETTES PAs with different lipid portion chain lengths on bulk collagen deposition by hCSFs after 7 days in culture (A), as well as the collagen produced per cell (B), using the Sirius-red/picric acid assay. Specifically, the C₈-ETTES and C₂₀-ETTES PAs were self-assembled in SFM, and their effect compared with that of C₁₆-ETTES. Mean±S.D., n=3 for all experiments; n.s., no statistically-significant differences between PAs with different lipid portion chain lengths and the C₁₆-ETTES control.

FIG. 10 shows the effect of fragment of the C₁₆-ETTES PA, C₁₆-ETTE, on hCSF proliferation at day 3 and 7 of culture. Specifically, C₁₆-ETTE was self-assembled in (A) SFM and (B) water, and its effect compared with that of C₁₆-ETTES. Quantification was performed using Alamar Blue assay. Mean±S.D., n=3 for all experiments; n.s., no statistically-significant differences between fragment and the C₁₆-ETTES control.

FIG. 11 shows the effect of fragment of the C₁₆-ETTES PA, C₁₆-ETTE, on bulk collagen deposition by hCSFs after 7 days in culture (A), as well as the collagen produced per cell (B), using the Sirius-red/picric acid assay. Specifically, C₁₆-ETTE was self-assembled in SFM, and its effect compared with that of C₁₆-ETTES. Mean±S.D., n=3 for all experiments; n.s., no statistically-significant differences between fragment and the C₁₆-ETTES control.

FIG. 12 shows the effect of C₁₆-ETTES fragments and PAs with different lipid portion chain lengths on collagen deposition by hASCs. The total amount of collagen deposited by hASCs after 7 days in culture with C₈-ETTES and C₂₀-ETTES variants, as well as with the C₁₆-ETTE fragment PA were assessed using the Sirius-red/picric acid assay, and compared with that of C₁₆-ETTES PA at 50 and 500 μM. Mean±S.D., n=3 for all experiments; n.s., no statistically-significant differences between ETTES fragments, different lipid chain lengths, and the C₁₈-ETTES control; * corresponded to statistically significant differences (p<0.05) between C₂₀-ETTES and control at highest concentration.

FIG. 13 shows the effect of 016-ETTES PAs with different lipid portion chain lengths and ETTES fragment on myoblast proliferation (A) and collagen deposition (B) at day 7 of culture when prepared with SFM or water at 50 μM. Cell quantification was performed using Alamar Blue assay; total amount of deposited collagen was assessed using the Sirius-red/picric acid assay. Mean±S.D., n=3 for all experiments; n.s., no statistically-significant differences between PAs with different lipid portion chain lengths, ETTES fragment and the C₁₆-ETTES control; * corresponded to statistically significant differences (p<0.05) between C₈-ETTES and control dissolved in water.

### DETAILED DESCRIPTION

A Method for Increasing Collagen Production and/or a Method for Inhibiting Cell Migration

In one aspect, the present invention provides a method for increasing production of an extracellular matrix protein in a cell. The method may comprise the step of contacting the cell with lipopeptides, wherein the lipopeptides substantially consist of ETTES lipopeptides.

In one example, the extracellular matrix protein is collagen.

Accordingly, in one aspect, the present invention provides a method for increasing collagen production in a cell. The method may comprise the step of contacting the cell with lipopeptides, wherein the lipopeptides substantially consist of ETTES lipopeptides.

In a further aspect, the present invention provides a method for inhibiting cell migration. The method may comprise the step of contacting the cell with lipopeptides, wherein the lipopeptides substantially consist of ETTES lipopeptides.

As mentioned elsewhere in the present specification, the inventors have found that culturing of cells in the presence of lipopeptides substantially consisting of ETTES lipopeptides may inhibit cell migration. The inventors believe that there may be a direct relationship between increasing collagen production and decreasing cell migration. Accordingly, it will be appreciated that the method for increasing collagen production may also be a method for inhibiting cell migration of a cell, and vice versa.

In one example, the cell may be a cultured cell. This example gives rise to a further aspect of the invention, which relates to a method for increasing collagen, the method comprising the step of culturing the cell in the presence of an aqueous medium comprising suspended therein lipopeptides, wherein the lipopeptides substantially consist of ETTES lipopeptides.

As used herein, the term “collagen” refers to the main protein of connective tissue that has a high tensile strength, and is found in most multicellular organisms. Collagen is a major fibrous protein, and it is also the nonfibrillar protein in basement membranes. It contains an abundance of glycine, proline, hydroxyproline, and hydroxylysine. In the context of the present disclosure, collagen includes any one or more types of collagen, whether native nor not, for example atelocollagen, insoluble collagen, collagen fibres, soluble collagen, and acid-soluble collagen. There are currently at least 28 types of collagen identified which are all encompassed herein.

Collagen may be for example fibrillar or non-fibrillar. Fibrillar collagen may be, for example, type I, II, III, V, Xl. Non-fibrillar collagen may be, for example, fibril associated collagen with interrupted triple helices (type IX, XII, XIV, XIX, XXI), short chain collagen (type VIII, X), basement membrane collagen (Type IV), multiplexin (XV, XVIII), membrane associated collagen with interrupted triple helices (type XIII, XVII), and collagen type VI and type VII. In one example, the collagen may be type I collagen or type III collagen.

It will be appreciated that the type of cells contacted with lipopeptides, wherein the lipopeptides substantially consist of ETTES lipopeptides, may influence the type of collagen produced. Examples of suitable cells are discussed elsewhere in the present specification. In one example, the cell may be in vivo or ex vivo. An ex vivo cell may be a cultured cell.

The term “increasing collagen production” as used herein refers to an increase in the amount of collagen biosynthesised and/or secreted by cells. The increase may be by an amount of at least 10%, at least 20%, at least 30%, at least 40%, at least 50%, at least 60%, at least 70%, at least 80%, at least 90%, at least 100%, or more as compared to a suitable control. For example, it may mean an increase by at least 110%, at least 120%, at least 130%, at least 140%, at least 150%, at least 160%, at least 170%, at least 180%, at least 190%, at least 200%, or more as compared to a suitable control.

In one example, the collagen production may be increased by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, or about 200%, as compared to a suitable control.

A suitable control may be, for example, a reference value based on the amount of collagen produced by cells not contacted with lipopeptides, wherein the lipopeptides substantially consist of ETTES lipopeptides.

The term “inhibiting cell migration” as used herein refers to a partial or complete reduction of the cell's movement from a starting position to a new position. The inhibited cell movement may be spontaneous migration and/or directional migration towards specific chemo-attractants.

In one example, cell migration may be inhibited if cell movement is reduced by least 10%, at least 20%, at least 30%, at least 40%, at least 50%, at least 60%, at least 70%, at least 80%, at least 90%, or more, as compared to a suitable control. A suitable control may be, for example, a reference value based on the migration distance of a cell not contacted with lipopeptides, wherein the lipopeptides substantially consist of ETTES lipopeptides. Methods of determining cell migration will be well known to those skilled in the art. Merely by way of example, cell migration may be determined by a cell migration assay as explained in the Examples section of the present specification.

The term “contacting” as used herein refers to bringing the cell into proximity with the lipopeptides, wherein the lipopeptides substantially consist of C₁₆-ETTES lipopeptides. Methods of contacting a cell will be known to those skilled in the art. It will be appreciated that methods of contacting may depend upon whether the cell is in vivo or ex vivo (such as a cultured cell).

In the context of an ex vivo cell, such as a cultured cell, “contacting” may mean providing the lipopeptides to a culture vessel (such as a tube, a flask, a dish or a plate comprising a plurality of wells, or the like) in which the cell is cultured. Providing the lipopeptides to the culture vessel in which the cell is cultured may also be referred to as “culturing a cell in the presence of lipopeptides”.

In the context of an in vivo cell, contacting may mean providing the lipopeptides to a subject. The subject may be provided the lipopeptides for therapeutic or non-therapeutic reasons. Examples of therapeutic and non-therapeutic uses of the lipopeptides substantially consisting of C₁₆-ETTES lipopeptides are discussed elsewhere in the present specification.

The term “culturing” as used herein refers to keeping cells in an artificial (e.g. in vitro or ex vivo) environment. Thus, “a cultured cell” is a cell that is kept in an artificial (e.g. in vitro or ex vivo) environment. Cells may be kept in an artificial environment without substantially increasing the cell number. Alternatively, cells may be kept in an artificial environment under conditions favouring proliferation, differentiation, and/or continued viability of the cells. Cells may be cultured for the purpose of cell bioprocessing. The term “cell bioprocessing” as used herein refers to producing a molecule of biological origin. The molecule of biological origin may be, for example, collagen. Accordingly, the method of increasing collagen production in a cell, may also be referred to as a method of bioprocessing a cell to produce collagen.

In the context of the present specification the cell can be an individual cell or a population of cells, or a tissue, organ (for example skin) or organ system. The cell may be eukaryotic (e.g., animal, plant and fungal cell) or prokaryotic (e.g., bacterial cell). The cell may be an animal cell. For example, the cell is mammalian (for example human, monkey, mouse, porcine, or bovine) or fish.

By way of example, the cell may be a stromal cell, myocyte, stromal progenitor cell, or adipose derived mesenchymal stem cell. Suitably, the cell may be a human stromal cell, human stromal progenitor cell or human adipose derived mesenchymal stem cell.

A stromal cell may be, for example, a corneal stromal cell or a fibroblast.

Merely by way of example, a mouse cell may be immortalised mouse myoblast cell.

The term “aqueous medium” as used herein refers to any liquid medium containing water. The aqueous medium may be cell culture medium, phosphate-buffered saline (PBS) or other saline solutions, or water. However, it will be appreciated that the term “aqueous medium” does not imply that water should always be the major constituent of the medium. The aqueous medium may be serum free.

The terms “cell culture medium” and “culture medium” (plural “media” in each case) refer to a nutritive solution for cultivating live cells and may be used interchangeably. The cell culture medium may be a complete formulation, i.e., a cell culture medium that requires no supplementation to culture cells, or may be an incomplete formulation, i.e., a cell culture medium that requires supplementation or may be a medium that may supplement an incomplete formulation or in the case of a complete formulation, may improve culture or culture results.

Various cell culture media will be known to those skilled in the art, who will also appreciate that the type of cells to be cultured may dictate the type of culture medium to be used.

Merely by way of example and not limitation, the culture medium may be selected from the group consisting of Dulbecco's Modified Eagle's Medium (DMEM), Ham's F-12 (F-12), Minimal Essential Medium (MEM), Basal Medium Eagle (BME), RPMI-1640, Ham's F-10, αMinimal Essential Medium (αMEM), Glasgow's Minimal Essential Medium (G-MEM), and Iscove's Modified Dulbecco's Medium(IMDM), or any combination thereof. Other media that are commercially available (e.g., from Thermo Fisher Scientific, Waltham, Mass.) or that are otherwise known in the art can be equivalently used in the context of this disclosure. Again, only by way of example, the media may be selected from the group consisting of 293 SFM, CD-CHO medium, VP SFM, BGJb medium, Brinster's BMOC-3 medium, cell culture freezing medium, CMRL media, EHAA medium, eRDF medium, Fischer's medium, Gamborg's B-5 medium, GLUTAMAX™ supplemented media, Grace's insect cell media, HEPES buffered media, Richter's modified MEM, IPL-41 insect cell medium, Leibovitz's L-15 media, McCoy's 5A media, MCDB 131 medium, Media 199, Modified Eagle's Medium (MEM), Medium NCTC-109, Schneider's Drosophila medium, TC-100 insect medium, Waymouth's MB 752/1 media, William's Media E, protein free hybridoma medium II (PFHM II), AIM V media, Keratinocyte SFM, defined Keratinocyte SFM, STEMPRO® SFM, STEMPRO® complete methylcellulose medium, HepatoZYME-SFM, Neurobasal™ medium, Neurobasal-A medium, Hibernate™ A medium, Hibernate E medium, Endothelial SFM, Human Endothelial SFM, Hybridoma SFM, PFHM II, Sf 900 medium, Sf 900 II SFM, EXPRESS FIVE® medium, CHO-S-SFM, AMINOMAX-II complete medium, AMINOMAX-C100 complete medium, AMINOMAX-C140 basal medium, PUB-MAX™ karyotyping medium, KARYOMAX bone marrow karyotyping medium, and KNOCKOUT D-MEM, or any combination thereof.

The cell culture medium may be serum-free. For example, the serum-free medium may be DMEM or F-12, or a combination thereof (DMEM-F12).

In the context of the present disclosure, the cell may be cultured in the presence of lipopeptides substantially consisting of ETTES lipopeptides for a time period suitable to increase the cell's collagen production and/or inhibit cell migration. In one example, the cell may be cultured in the presence of the lipopeptides for at least 1 hr, at least 2 hrs, at least 3 hrs, at least 4 hrs, at least 5 hrs, at least 6 hrs, at least 7 hrs, at least 8 hrs, at least 9 hrs, at least 10 hrs, at least 12 hrs, at least 14 hrs, at least 16 hrs, at least 18 hrs, at least 20 hrs, at least 22 hrs, at least 24 hrs, or more. For example, the cell may be cultured in the presence of the lipopeptides for at least 36 hrs, at least 48 hrs, at least 60 hrs, at least 72 hrs, at least 84 hrs, at least 96 hrs, at least 108 hrs, or at least 120 hrs, or more. In one example the cells may be cultures from about 1 hr to about 120 hrs, or from about 24 hrs to about 96 hrs.

By the same token, the cell may be cultured in the presence of lipopeptides substantially consisting of ETTES lipopeptides, wherein the lipopeptides are at a concentration suitable to increase the cell's collagen production and/or inhibit cell migration. In one example, the cell may be cultured at an ETTES lipopeptide concentration of about 0.1 μM, about 0.2 μM, about 0.3 μM, about 0.4 μM, about 0.5 μM, about 0.6 μM, about 0.7 μM, about 0.8 μM, about 0.9 μM, about 1 μM, about 2 μM, about 3 μM, about 4 μM, about 5 μM, about 6 μM, about 7 μM, about 8 μM, about 9 μM, or more. For example, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, or about 100 μM, or more. For example, about 150 μM, about 200 μM, about 250 μM, about 300 μM, about 350 μM, about 400 μM, about 450 μM, about 500 μM, about 550 μM, about 600 μM, about 650 μM, about 700 μM, about 750 μM, about 800 μM, about 850 μM, about 900 μM, about 950 μM, about 1000 μM or more.

For example, the cell may be cultured in the presence of the ETTES lipopeptides, wherein the ETTES lipopeptides are at a concentration from about 0.1 μM to about 1000 μM, from about 0.5 μM to about 750 μM, or from about 1 μM to about 500 μM. For example, the cell may be cultured in the presence of the lipopeptides, wherein the lipopeptides are at a concentration from about 0.1 μM to about 10 μM or from about 0.5 μM to about 5 μM.

Methods of determining a suitable concentration will be known to those skilled in the art. It will be appreciated that the mentioned concentrations of lipopeptides may be applicable in the context of the pharmaceutical composition described herein. In other words, the pharmaceutical composition described herein, may comprise lipopeptides substantially consisting of ETTES lipopeptides, wherein the lipopeptides are at these concentrations.

The term “suspended” as used herein means that the lipopeptides are fully or partially submerged in the aqueous medium. The submerged lipopeptides may be assembled into supramolecular structures, non-assembled lipopeptide molecules, or partially assembled lipopeptide molecules.

In an example where the lipopeptides are in the form of supramolecular structures, the supramolecular structures may have been assembled in any suitable aqueous medium, for example water, or cell culture medium. The supramolecular structure may have any suitable topology. The type of medium in which the supramolecular structure has been assembled may influence the topology of the supramolecular structure.

For example, when the supramolecular structure is assembled in a solvent having an ionic strength that is greater than the ionic strength of distilled water (such as cell culture medium), the supramolecular structure may have a globular topology resulting from aggregated structures. Herein such a supramolecular structure is referred to as a “fibrillar supramolecular structure”, “globular supramolecular structure” or “aggregated supramolecular structure”. Such a fibrillar supramolecular structure is topologically distinct from lipopeptide structures defined in the art which are assembled in water, and form fibrillar nanotapes having a width from about 5 to about 50 nm, rather than globules. The inventors believe that this new structure is formed as a result of the ionic strength of the solvent in which the lipopeptides self-assemble. The inventors hypothesise that the increased ionic strength of the solvent (compared to water) generates electrostatic attraction between the lipopeptides which changes how the lipopeptides assemble. These findings give rise to further aspects of the invention, which provide a fibrillar supramolecular structure, as well as a method of producing the fibrillar supramolecular structure. These aspects are described elsewhere in the present specification.

By contrast, when the supramolecular structure is assembled in distilled water, the supramolecular structure does not have a globular topology. Such a supramolecular structure may have a fibre-like or fibrillar nanotape topology as known in the art. The present inventors have surprisingly found that in the context of ETTES lipopeptides, such a fibre-like supramolecular structure is particularly useful in the context of increasing collagen production in a cell, such a myoblast cell, for example a mouse myoblast cell. On other hand, a fibrillar supramolecular structure may be particularly useful in the context of increasing collagen production in a cell such as a stromal cell, a stromal progenitor cell and an adipose derived mesenchymal stem cell. The stromal cell may be for example a corneal stromal cell or a fibroblast, for example human corneal stromal cell or a fibroblast. By “non-assembled lipopeptide molecules” it is meant that substantially all of the lipopeptides are in the aqueous medium as individual molecules. By “partially assembled lipopeptides” it is meant that some of the lipopeptides have assembled into supramolecular structures whereas others are in the aqueous medium as individual molecules. When the lipopeptides are in the aqueous medium as individual molecules, some or all of the molecules may be dissolved in the aqueous medium.

### Lipopeptides

The term “lipopeptide” as used herein refers to an amphiphilic molecule comprising or consisting of a lipid portion and an amino acid portion. The terms “lipopeptide”, “amphiphilic molecule”, “peptide amphiphile” and “PA” are used interchangeably herein.

The amphiphilic properties enable a plurality of lipopeptides to self-assemble into the supramolecular structure. Lipopeptides are well known and their self-assembly properties are well characterised in the art (see for example Cui H. et al., Biopolymers, 2010; 94(1): 1-18). Appropriate lipopeptides may therefore easily be identified by a person of skill in the art e.g. by testing their propensity to self-assemble under certain conditions and form supramolecular structures. Lipopeptide self-assembly and corresponding c.a.c. can be evaluated by the Thioflavin (ThT) and pyrene (Pyr) fluorescence spectroscopy methods. Fluorescence spectra are recorded with a Fluorescence Spectrometer. For the ThT assay, the spectra are typically recorded from 460 to 600 nm using an excitation wavelength λₑₓ=440 nm and the lipopeptide dissolved in a 4-5×10⁻³% (w/v) ThT solution. For the Pyr assay, the spectra are typically recorded from 360 to 550 nm using an excitation wavelength λₑₓ=339 nm. Pyr assays are performed using a 1-1.5×10⁻⁵% (w/v) Pyr solution as a diluent. The Florescence intensity is plotted against a log of the lipopeptide concentration. The inflection point for the data denotes a change of environment for the ThT/Pyr molecule and is used to identify the c.a.c.

A lipopeptide nanostructure can be evaluated by cryo-transmission electron microscopy (cryo-TEM) using a field-emission cryo-electron microscope (e.g. JEOL JEM-3200FSC), AFM, or small-angle X-ray scattering. For cryo-TEM, vitrified specimens are prepared onto holey carbon copper grids with 3.5 μm hole size. A lipopeptide solution is applied to the grid and then vitrified in a 1/1 mixture of liquid ethane and propane at −180° C. The cryo-electron microscope is operated at −187° C. during the imaging. Lipopeptide solutions are heated from −187° C. to −60° C. at ˜10-5 Pa, before being imaged at −187° C. The heating process from −187 to −60° C., equivalent to a freeze drying process in the microscope, allows for the sublimation of the ice from the sample and removes the vitrified water. Images are taken using bright-field mode and zero-loss energy filtering (omega type) with a slit width 20 eV. Micrographs are recorded using a CCD camera (e.g. Gatan Ultrascan 4000).

The amino acid portion of the lipopeptide may be a natural or synthetic amino acid sequence. A natural amino acid sequence is one that exists in nature and encodes a protein or a fragment thereof. The natural amino acid sequence may encode a human, animal, plant, fungal, Protista, Archaea, and/or bacterial protein or fragment thereof. The fragment may comprise, for example, from about 3 to about 40 amino acids, such as from about 3 to about 20, or from about 3 to about 10 amino acids. The synthetic amino acid may be, for example, a variant of the natural amino acid sequence.

In the context of the present disclosure, an ETTES lipopeptide is a lipopeptide in which the amino acid portion comprises or consists of the amino acid sequence ETTES or a fragment or variant thereof. The term “ETTES lipopeptide” therefore encompasses all lipopeptides that comprise the ETTES amino acid sequence, as well as those with ETTES fragment sequences or ETTES variant sequences. For the avoidance of doubt, ETTES lipopeptides therefore do not necessarily have the fully ETTES amino acid sequence, but may comprise a fragment of the ETTES sequence, or a variant sequence instead. All such lipopeptides are encompassed by the term “ETTES lipopeptide” as used herein.

Without wishing to be bound by hypothesis, the inventors believe that the effect of the ETTES lipopeptide on collagen production and/or cell migration may be, at least partially, due to the negative charge of the ETTES amino acid sequence. Thus, in one example, the ETTES fragment or variant may have a negative charge. In one example, the ETTES fragment or variant may have substantially the same negative charge as the ETTES amino acid sequence.

An ETTES fragment is a peptide that is shorter than the corresponding ETTES amino acid sequence. An ETTES fragment may share 100% identity with the portion of the ETTES amino acid sequence that it corresponds to. The fragment may be at least 3 amino acid residues in length. For example, the fragment may be 3 or 4 amino acid residues in length. For example, the fragment may have a sequence selected from the group consisting of ETT, TTE, TES, ETTE and TTES. Suitably, the fragment may have the sequence ETTE. As mentioned in the Examples section, the inventors have shown that peptides comprising fragments of ETTES amino acid sequences may also increase collagen production while increasing, maintaining, or reducing cell proliferation, depending on cell type and formulation method. Suitably, when the amino acid portion comprises or consists of the amino acid sequence ETTE, the lipid portion may be C16. Such a lipopeptide may be referred to as a C₁₆-ETTE lipopeptide.

As used herein, the term “variant” refers to an amino acid sequence in which one or more amino acids have been replaced by different amino acids as compared to the corresponding amino acid sequence. Accordingly, an ETTES variant refers to an amino acid sequence in which one or more amino acids have been replaced by different amino acids as compared to the ETTES amino acid sequence. For example, the variant may be selected from the group consisting of ETETS, TTEES, SEETT, SETET, TESTE, or any one of the aforementioned variants wherein any one or more glutamate (E) residues is substituted with an aspartic acid (D) residue.

It is well understood in the art that some amino acids may be changed to others with broadly similar properties without changing the nature of the activity of the peptide (conservative substitutions). Generally, the substitutions which are likely to produce the greatest changes in a peptide's properties are those in which (a) a hydrophilic residue (e.g., Ser or Thr) is substituted for, or by, a hydrophobic residue (e.g. Leu, lie, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a bulky side chain (e.g., Phe or Trp) is substituted for, or by, one having a smaller side chain (e.g., Ala, Ser) or no side chain (e.g., Gly).

In one example, the amino acid portion does not comprise a cell adhesion moiety, meaning that the amino acid portion will not comprise an extracellular matrix protein motif, or a fragment or variant thereof, that is involved in cell adhesion.

In the context of the present disclosure, the fragment or variant may substantially retain the biological function of the corresponding sequence. For example, when the corresponding sequence is ETTES, the fragment or variant may substantially retain the biological function of the ETTES sequence.

The term “biological function” as used herein may refer to the ability to increase cell collagen production and/or inhibit cell migration. This biological function is particularly relevant to lipopeptides, wherein the lipopeptides substantially consist of ETTES lipopeptides, as well as fragments or variants thereof.

By “substantially retains” biological function, it is meant that the fragment or variant retains at least about 50%, 60%, 75%, 85%, 90%, 95%, 97%, 98%, 99%, or more, of the biological function of the corresponding ETTES sequence. Indeed, the fragment or variant may have a higher biological function than the corresponding ETTES sequence. The fragment or variant may have 110%, 120%, 130%, 140%, 150%, 160%, 170%, 180%, 190%, 200%, or more, of the biological function of the corresponding ETTES sequence. The biological function may be, for example, the ability to increase collagen production in a cell and/or inhibit cell migration. Methods of determining whether a fragment or variant has the ability to increase collagen production in a cell and/or inhibit cell migration will be known to those skilled in the art. Merely by way of example, such examples include collagen staining, total collagen assay, or cell migration assay.

The amino acid portion of the lipopeptide (for example ETTES lipopeptide) may comprise or consist of one or more amino acid sequences, fragments, and/or variants thereof.

For example, the amino acid portion of the ETTES lipopeptide may comprise or consist of 1, 2, 3, 4, 5 or more ETTES sequences, fragments, and/or variants thereof. The ETTES sequences, fragments, and/or variants thereof may be in tandem or may be spatially separated e.g. by other amino acids or linkers within the amino acid portion of the lipopeptide.

In an example where the amino acid portion of the lipopeptide comprises more than one amino acid sequence, fragment, and/or variant thereof, some or all of the sequences, fragments, and/or variants may be same. Alternatively, some or all of the peptides, fragments, and/or variants may be different.

Accordingly, in the context of an ETTES lipopeptide, in an example where the amino acid portion of the lipopeptide comprises more than ETTES sequence, fragment, and/or variant thereof, some or all of the ETTES sequences, fragments, and/or variants may be same. Alternatively, some or all of the ETTES sequences, fragments, and/or variants may be different.

The lipid portion of the lipopeptide (for example ETTES lipopeptide) may be linear, branched or cyclic. For example, the lipid portion may be linear.

The lipid portion may comprise a hydrophobic carbon chain of 6 to 24 carbon atoms (for example 8 to 20 carbon atoms). The lipid portion may therefore comprise a carbon chain of 6, 7, 8, 9, 10, 11, 12, 13, 14, 15, 16, 17, 18, 19, 20, 21, 22, 23, 24 or more carbon atoms. For example, the lipid portion will comprise a carbon chain of 8, 16 or 20 carbon atoms. It will be appreciated that, when a lipid portion is referred to as, for example, C16 or C18, it means that the lipid portion comprises carbon chain of 16 or 18 carbon atoms, respectively. By way of example and not limitation, the lipid portion may comprise or consist of dodecanoic acid (lauric acid), tetradecanoic acid (myristic acid), hexadecanoic acid (palmitic acid), octadecenoic acid (stearic acid), oleic acid, linoleic acid, and linolenic acid.

The lipid portion may be saturated or unsaturated.

The lipid portion and amino acid portion of the lipopeptide (for example ETTES lipopeptide) may be attached directly or indirectly. By attached directly, it is meant that the lipid and peptide portions are not separated by a linker. For example, the lipid and amino acid portion may be covalently coupled. By attached indirectly it meant that the lipid and peptide portions are separated by a linker.

By way of example, in the context of an ETTES lipopeptide, the lipopeptide may comprise a lipid portion which comprises or consists of a carbon chain of 8, 16, or 20 carbon atoms. An ETTES lipopeptide having a lipid portion which comprises or consists of a carbon chain of 16 carbon atoms may be herein referred to as a C₁₆-ETTES lipopeptide.

The term “substantially consists of” as used herein, refers to the proportion of ETTES lipopeptides to non-ETTES lipopeptides (i.e. lipopeptides that do not comprise an ETTES sequence, or fragment or variant thereof) within the aqueous medium, the supramolecular structure, or pharmaceutical composition as described herein. By “substantially consists of” it is meant that the majority of lipopeptides are ETTES lipopeptides. For example, the aqueous medium or the supramolecular structure may substantially consist of ETTES lipopeptides when ETTES lipopeptides account for at least 90%, at least 91%, at least 92%, at least 93%, at least 94%, at least 95%, at least 96%, at least 97%, at least 98%, at least 99%, or 100% of all lipopeptides in the aqueous medium or supramolecular structure. In one example, the ETTES lipopeptides accounts for 100% of the lipopeptides in the aqueous medium or the supramolecular structure.

### A Pharmaceutical Composition and Uses Thereof

Also provided by the present invention is a pharmaceutical composition comprising lipopeptides, wherein the lipopeptides substantially consist of ETTES lipopeptides.

The lipopeptides in the pharmaceutical composition may be in the form of non-assembled lipopeptide molecules, partially assembled lipopeptides, or supramolecular structures. In examples where the lipopeptide are non-assembled or partially assembled, the lipopeptide molecules may be dissolved in the pharmaceutical composition. In such an example, the pharmaceutical composition may be substantially transparent.

In some examples, the composition further comprises a pharmaceutically acceptable diluent, carrier or excipient. The composition may further routinely contain pharmaceutically acceptable concentrations of salt, buffering agents, preservatives (for example antioxidants), supplementary immune potentiating agents such as adjuvants and cytokines and optionally other therapeutic agents.

The phrase “pharmaceutically acceptable” is employed herein to refer to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio.

Diluents are diluting agents. Pharmaceutically acceptable diluents are well known in the art. A suitable diluent is therefore easily identifiable by one of ordinary skill in the art.

Carriers are non-toxic to recipients at the dosages and concentrations employed and are compatible with other ingredients (such as the lipopeptides) of the composition. The term “carrier” denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art.

Excipients are natural or synthetic substances formulated alongside an active ingredient (e.g. the lipopeptides as provided herein), included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like.

Adjuvants are pharmacological and/or immunological agents that modify the effect of other agents in a formulation. Pharmaceutically acceptable adjuvants are well known in the art. A suitable adjuvant is therefore easily identifiable by one of ordinary skill in the art.

Preservatives may be antioxidants. As antioxidants may be mentioned thiol derivatives (e.g. thioglycerol, cysteine, acetylcysteine, cystine, dithioerythreitol, dithiothreitol, glutathione), tocopherols, butylated hydroxyanisole, butylated hydroxytoluene, sulfurous acid salts (e.g. sodium sulfate, sodium bisulfite, acetone sodium bisulfite, sodium metabisulfite, sodium sulfite, sodium formaldehyde sulfoxylate, sodium thiosulfate) and nordihydroguaiareticacid. Suitable preservatives may for instance be phenol, chlorobutanol, benzylalcohol, methyl paraben, propyl paraben, benzalkonium chloride and cetylpyridinium chloride.

The pharmaceutical compositions described above may be suitable for use in therapeutic and non-therapeutic (for example cosmetic) applications.

The pharmaceutical composition may be for administration to a subject by any suitable route by which an effective amount of the pharmaceutical composition may be provided to the subject. Merely by way of example a suitable route of administration may be dermal, transdermal, intra-articular, subcutaneous, intramuscular, or intravenous.

The pharmaceutical composition may be in the form of an ointment, gel, cream, liquid, powder, or liniment.

In one example, the ointment, gel, cream, liquid, powder or liniment, may be applied onto, absorbed, adsorbed or incorporated into a bandage, a scaffold (for example a sheet suitable for use as a sheet mask), or sustained-release matrix (for example hydrogel).

In one example, the pharmaceutical composition may be a sterile pharmaceutical composition. It will be appreciated that a sterile pharmaceutical composition is particularly useful in the context of a composition for intra-articular, subcutaneous, intramuscular, or intravenous administration.

A sterile pharmaceutical composition may be created, for example, by filtration through sterile filtration membranes, prior to or following lyophilisation and/dissolving of the lipopeptides. The lipopeptides may be stored in lyophilised form or in a suitable aqueous medium.

A sterile pharmaceutical composition comprising the lipopeptides may be placed into a container having a sterile access port, for example, an intravenous solution bag or vial having an adapter that allows retrieval of the formulation, such as a stopper pierce-able by a hypodermic injection needle.

A sterile pharmaceutical composition comprising the lipopeptides suitable for intra-articular, subcutaneous, intramuscular, or intravenous delivery may be formulated according to conventional pharmaceutical practice as described in Remington: The Science and Practice of Pharmacy (20^{th} ed., Lippincott Williams & Wilkens Publishers (2003)). For example, dissolution or suspension of the lipopeptides in a vehicle such as water, PBS, naturally occurring vegetable oil like sesame, peanut, or cottonseed oil or a synthetic fatty vehicle like ethyl oleate or the like may be desired. Buffers, preservatives, antioxidants and the like can be incorporated according to accepted pharmaceutical practice.

In an example, the pharmaceutical composition comprising the lipopeptides may be for the sustained release of the lipopeptides. Such a pharmaceutical composition may comprise semipermeable matrices of solid hydrophobic polymers containing the lipopeptides, which matrices are in the form of shaped articles, films or microcapsules. Examples of sustained-release matrices include polyesters, hydrogels, copolymers of L-glutamic acid and gamma ethyl-L-glutamate, non-degradable ethylene-vinyl acetate, degradable lactic acid-glycolic acid copolymers such as the LUPRON Depot™ (injectable microspheres composed of lactic acid-glycolic acid copolymer and leuprolide acetate), and poly-D-(−)-3-hydroxybutyric acid.

The term “hydrogel” as used herein refers a structure made from cross-linked polymers. The hydrogel may be insoluble in water but may be capable of absorbing and retaining large quantities of water to form a stable, often soft and pliable, structure. The hydrogel may comprise internal pores. The pores may be penetrable by lipopeptides, such that the lipopeptides may be partially or fully retained within the hydrogel. The lipopeptides in the hydrogel may be in the form of non-assembled lipopeptide molecules, partially assembled lipopeptides, or supramolecular structures.

In one aspect, the pharmaceutical composition described herein is for use in therapy.

In further aspect, the present invention provides use of a pharmaceutical composition comprising lipopeptides, wherein the lipopeptides substantially consist of ETTES lipopeptides.

In one example, the pharmaceutical composition may be for use in the treatment of a collagen deficiency disease and/or for use in enhancing wound healing.

Accordingly, in a further aspect the present invention provides a pharmaceutical composition for use in the treatment of a collagen deficiency disease and/or for use in enhancing wound healing, wherein the composition comprises lipopeptides, wherein the lipopeptides substantially consist of ETTES lipopeptides.

In yet a further aspect, the present invention provides a method of treating a collagen deficiency disease and/or a method of enhancing wound healing in a subject, the method comprising the step of providing to the subject a therapeutically effective amount of a pharmaceutical composition comprising lipopeptides, wherein the lipopeptides substantially consist of ETTES lipopeptides.

A “collagen deficiency disease” refers to any disease in which a subject has or is at risk of having a reduced amount of collagen. The subject may have a reduced amount of collagen, for example due to the subject's cells not producing a sufficient amount of collagen. Insufficient production of collagen may be for example due to a genetic defect.

A collagen deficiency disease may be for example Ehlers-Danlos syndrome, Marfan's syndrome, Osteogenesis imperfecta, brittle bone disease, or collagen vascular disease. Examples of collagen vascular diseases include lupus, rheumatoid arthritis, systemic sclerosis, temporal arteritis. Other diseases where the patient has or is at risk of having a reduced amount of collagen will be known to the skilled person. The collagen deficiency disease may be a primary or secondary disease.

It will be appreciated that when reference is made to a reduced amount of collagen it is meant that the amount of collagen produced by a cell is lower than compared to a suitable control. A suitable control may be for example a reference value derived from collagen levels produced by cells from an individual not suffering from a collagen deficiency disorder.

In one example, the pharmaceutical composition may be for use in inhibiting cell migration. Inhibiting cell migration may be desirable, for example, in the context of cancer.

Accordingly, in one aspect, the present invention provides a pharmaceutical composition for use in the treatment of cancer, wherein the composition comprises lipopeptides, wherein the lipopeptides substantially consist of ETTES lipopeptides.

As used herein, the term “cancer” refers to a single or cluster of overgrowing cells, characterised by upregulated cell growth, and replication, reduced cell differentiation, and/or the ability to metastasize to other parts of the body. In one example, the cancer may be a solid cancer. Merely by way of example, a solid cancer may be selected from the group consisting of skin cancer, oesophageal cancer, and oral cancer (such as oral squamous cell carcinoma).

In the context of the present disclosure the terms “treat”, “treating” or “treatment” refer to a clinical improvement of the relevant disease in a subject with the disease. Such a clinical improvement may be demonstrated by an improvement of the pathology and/or symptoms associated with the diseases. Symptoms associated with a collagen deficiency disease may include fatigue, muscle weakness, body aches, joint pain, and/or a skin rash. Symptoms associated with cancer may include, for example, fever, fatigue, weight loss. In the context of cancer, clinical improvement of the pathology may be demonstrated by one or more of the following: reduced biomarker levels in the subject, increased time to regrowth of cancer upon stopping of treatment, lack of regrowth of cancer upon stopping treatment, decreased tumour invasiveness, reduction or complete elimination of metastasis, increased cancer cell differentiation, or increased survival rate.

The term “treatment” encompasses not only the therapeutic use of lipopeptides in a subject with the symptoms of a collagen deficiency disease, but also the use of lipopeptides in the treatment of a subject who does not exhibit the symptoms of the disease. Such uses may be of particular relevance to an asymptomatic subject, for example, known to carry a mutation which increases the subject's likelihood of developing a collagen deficiency disease.

As used herein, the term “wound” refers to damage or loss to any one or combination of skin layers caused by cuts, incisions (including surgical incisions), abrasions, microbial infections, diseases or disorders, necrotic lesions, lacerations, fractures, contusions, burns and amputations. Non-limiting examples of wounds can include bed sores, thin dermis, bullous skin disease, and other cutaneous pathologies, such as subcutaneous exposed wounds that extend below the skin into the subcutaneous tissue. In some instances, a subcutaneous exposed wound may not affect underlying bones or organs.

The phrase “enhancing wound healing” as used herein refers to improving the natural cellular processes of tissue repair such that healing is faster, and/or the resulting healed area has less scaring, and/or the wounded area possesses tissue strength that is closer to that of uninjured tissue, and/or the wounded tissue attains some degree of functional recovery.

The term “providing” as used herein encompasses any techniques by which the subject receives a therapeutically effective amount of the pharmaceutical composition comprising the lipopeptides. Exemplary routes of administration are discussed elsewhere in the present specification. It will be appreciated that preferred routes of administration will depend on the diseases, or type and/or location of wound.

The term “therapeutically effective amount” as used herein, refers to an amount of the pharmaceutical composition, that when provided to the subject, it is sufficient to increase the amount of collagen in a subject and thereby treat a collagen deficiency disease, enhance wound healing in a subject, and/or inhibit cell migration.

It will be appreciated that the therapeutically effective amount will vary depending on various factors, such as the subject's body weight, sex, diet and route by which the lipopeptides are administered. A therapeutically effective amount may be provided to the subject in a single dose or in multiple doses.

As used herein, the term “subject” refers to any individual who may benefit from increased collagen production and/or inhibited cell migration. The subject may be a human subject.

An individual who may benefit from increased collagen production may have symptoms associated with a collagen deficiency disease, such as fatigue, muscle weakness, body aches, joint pain, and/or a skin rash. Alternatively, the subject may be asymptomatic but at risk of developing such symptoms. In one example, the subject may be an individual diagnosed with a collagen deficiency disease, for example collagen vascular disease, such as lupus, rheumatoid arthritis, systemic sclerosis, or temporal arteritis. Alternatively, the individual may have a wound. An individual who may benefit from inhibited cell migration may be diagnosed with cancer.

As mentioned above, the present invention provides use of a pharmaceutical composition comprising lipopeptides, wherein the lipopeptides substantially consist of ETTES lipopeptides. The use may be therapeutic (as described hereinabove) or non-therapeutic (for example cosmetic).

As used herein, the terms “cosmetic” refers to interventions performed with the intention of addressing (e.g. improving, preventing or regulating) a non-pathological condition in a subject, such as the signs of aging on the subject's skin. Accordingly, cosmetic treatments may be used to restore or improve the appearance of a subject. The subject's appearance may be restored or improved for example by reducing or preventing skin wrinkles, reducing or preventing skin hyperpigmentation and/or increasing skin elasticity or preventing loss of skin elasticity. It will be appreciated that these effects may be achieved by increasing the amount of collagen production by the subject's cells. Thus, in the context of cosmetic uses, the subject may be any individual wishing to restore or improve their appearance.

It will be appreciated that non-therapeutic uses are not limited to cosmetic uses. Other uses of the lipopeptides substantially consisting of ETTES lipopeptides, or compositions comprising lipopeptides substantially consisting of ETTES lipopeptides are also contemplated herein. Merely by way of example, lipopeptides substantially consisting of ETTES lipopeptides or compositions comprising such lipopeptides may be used in an in vitro method for producing tissue. Such a method may be particularly useful in the context of producing meat or other nutritional products. In one example, the in vitro produced tissue is non-human. It will be appreciated that the usefulness of the ETTES lipopeptides or compositions comprising such lipopeptides as described herein, in producing tissue may be due to the ability of the lipopeptides to increase collagen production. Such an increase in collagen production may therefore enhance tissue (for example meat) production as compared to methods that don't employ such lipopeptides. Suitably, the ETTES lipopeptides or compositions comprising such lipopeptides as described, may be used in a method for producing tissue in vitro (such as meat), wherein the method comprises increasing collagen production in a cell by culturing the cell with lipopeptides, wherein the lipopeptides substantially consist of ETTES lipopeptides. The ETTES lipopeptides or compositions comprising such lipopeptides as described herein may be used in other settings, where an increase in collagen production is desired.

### “A Supramolecular Structure”

The term “supramolecular structure” are used herein refers to an aggregate comprising lipopeptides, wherein the lipopeptides substantially consist of ETTES lipopeptides.

The aggregated lipopeptides may form a plurality of fused fibrils. A supramolecular structure formed from a plurality of fused fibrils may be interchangeably referred to herein as a “fibrillar supramolecular structure” or a “globular supramolecular structure”. Such a supramolecular structure has a novel, globular topology.

The present inventors have surprisingly found that this novel, globular topology is formed by lipopeptides that have been assembled in a solvent having an ionic strength that is greater than the ionic strength of distilled water. By contrast, a supramolecular structure that is assembled in distilled water may have a fibre-like topology.

These findings give rise to further aspects of the invention.

Accordingly, in one aspect the present invention provides a fibrillar supramolecular structure comprising lipopeptides, wherein the lipopeptides substantially consist of ETTES lipopeptides.

In a further aspect, the present invention provides a method of producing a fibrillar supramolecular structure comprising lipopeptides, wherein the lipopeptides substantially consist of ETTES lipopeptides, the method comprising dissolving the lipopeptides in a solvent having an ionic strength that is greater than the ionic strength of distilled water to produce the supramolecular structure.

Fused fibrils forming the fibrillar supramolecular structure can be identified, for example, using cryo-transmission electron microscopy (cryo-TEM), AFM, small-angle X-ray scattering, or other methods also well known to those skilled in the art, or described elsewhere in the present specification.

The fibrils may be nanofibers, filaments, tapes, tubes, twisted fibres, twisted filaments, twisted tapes, twisted tubes, or networks, or combinations thereof. The structural characteristics of a fibril are well known in the art (see for example the following reviews by I. W. Hamley (Soft Matter, 2011, 7: 4122) and Stupp et al. (Faraday Discussions, 2013, 166: 9-30)).

Typically, a fibril may be in the region of about 40-290 nm wide and/or about 150-2500 nm long. The structure may be made up of uniformly and/or non-uniformly shaped fibrils. Within the structure, the fibrils may be of substantially the same size or of different size.

A plurality of fibrils present in the structure are fused together. For example, at least two, three, four, five, six, seven, eight, nine, ten, or more fibrils may be fused together to form the supramolecular structure.

The fibrillar supramolecular structure may form dense globular deposits. The globular deposits may have a diameter of at least 200 nm. For example, the globular deposits may have a diameter of at least 300, at least 400, at least 500, at least 600, at least 700, at least 800 etc nm. In one example, they have a diameter of from about 200 to about 800 nm wide.

The fibrillar supramolecular structure described herein may be produced by the method comprising the step of dissolving the lipopeptides, wherein the lipopeptides substantially consist of ETTES lipopeptides, in a solvent having an ionic strength that is greater than the ionic strength of distilled water. A solvent having an ionic strength that is greater than the ionic strength of distilled water may be referred to herein as “a solvent with high ionic strength”.

The fibrillar supramolecular structures generated herein using solvents with high ionic strength may have a higher fibril density than those generated in the art using the same lipopeptides with water. It will be appreciated that density can be determined using cryo-transmission electron microscopy (cryo-TEM) or AFM, by analysing the total area occupied by structures formed in different conditions. Details of other appropriate methods are also well known in the art.

In one example, the fibrillar supramolecular structures generated herein using solvents with high ionic strength may have a density of fibrils that is at least 10%, at least 20%, at least 30%, at least 40%, at least 50% higher than the density of fibrils in a supramolecular structure generated using the same lipopeptides with water. For example, the fibrillar supramolecular structures generated herein may have a density of fibrils that is at least 40% higher than the density of fibrils in a supramolecular structure generated using the same lipopeptides with water.

As will be appreciated by a person of skill in the art, in the context of this specification, when a comparison is made to a supramolecular structure generated using water as the solvent, the water that is meant is distilled water. Accordingly, any reference to “water” solvents herein refer to distilled water.

In one example, the fibrillar supramolecular structure of the present invention may be in an aqueous medium or on/in a surface that is suitable for cell culture. This example gives rise to further two aspects of the invention.

Accordingly, in one aspect provided herein is an aqueous medium comprising the fibrillar supramolecular structure described herein. Examples of a suitable aqueous medium are provided elsewhere in the present specification. Merely by way of example, the aqueous medium may be cell culture medium or water. The cell culture medium may be serum free. It will be appreciated that when the aqueous medium is water, the fibrillar supramolecular structure would have been assembled in a solvent having a high ionic strength and subsequently transferred to the water. If on the other hand, the aqueous medium is, for example, cell culture medium, the supramolecular structure may have been assembled in the cell culture medium it is provided in.

In a further aspect, provided herein is a surface, wherein immobilised in or on the surface are lipopeptides, wherein the lipopeptides substantially consist of ETTES lipopeptides.

The term “surface” as used herein refers to an area on which cells may be cultured. The surface can be 2-dimensional (2D) or 3-dimensional (3D). An example of a 2D surface is a cover slip, or a surface of a culture vessel, such as a tube, a flask, a dish or a plate comprising a plurality of wells. The culture vessel may be a glass, plastic, or metal container that can provide an aseptic environment for culturing cells. An example of a 3D surface is a scaffold, such as a polystyrene scaffold (eg. Alvetex™) or a gel scaffold (eg. hydrogel).

The fibrillar supramolecular structure may be immobilised on the surface so as to provide a surface that is coated with the supramolecular structure. The surface may be coated partially or completely. Methods of coating surfaces with supramolecular structures are generally known in the art. By way of example, a surface may be coated by drop-spotting on the surface and homogenously distributing a solution comprising the lipopeptides (such as ETTES lipopeptides), followed by drying the surface to form a thin film of self-assembled fibrillar supramolecular structure. In an example, the fibrillar supramolecular structure may be immobilised on a 2D surface, such as a cover slip, or a surface of a culture vessel such as a tube, a flask, a dish or a plate comprising a plurality of wells.

The fibrillar supramolecular structure may be immobilised in the surface for cell culture. By “in the surface” is meant that the globular supramolecular structure is incorporated into the surface, such that it is partially or fully encapsulated by the surface. Methods for incorporating into a surface a supramolecular structure are also known in the art. For example, the lipopeptides that form the fibrillar supramolecular structure may be added to the solution from which the surface (such as a 3D scaffold) is made from.

It will be appreciated that in some examples, the fibrillar supramolecular structure described herein may, in fact, itself be the surface for cell culture. In such an example, the structure may be in an aqueous medium or may be immobilised in or on the surface.

As mentioned, in one aspect, the present invention provides a method of producing a fibrillar supramolecular structure comprising lipopeptides, wherein the lipopeptides substantially consist of ETTES lipopeptides. The method comprises the step of dissolving the lipopeptides in a solvent having an ionic strength that is greater than the ionic strength of distilled water to produce the supramolecular structure.

The term “dissolving” refers to incorporating the lipopeptides into a liquid solvent, so as to form a solution. The terms “dissolving” and “solubilising” (and variants thereof) are used interchangeably herein. The lipopeptides (for example ETTES lipopeptides) to be dissolved may be lyophilised. It will be appreciated that dissolving the lipopeptides may be aided by mixing. Thus, in the context of the present disclosure, the step of dissolving may comprise the step of mixing. Mixing may comprise, for example, vortexing, sonicating, rotating, and/or irrigating the solvent comprising the lipopeptides. The step of mixing may be carried out until the lipopeptides are dissolved in the solvent so as to form a substantially transparent solution.

By “substantially transparent” it is meant the lipopeptides have dissolved to the extent that they are no longer visible by eye (e.g. at a distance of 30 cm by a person with 20/20 vision). In other words, a substantially transparent solution is one that is optically clear.

Merely by way of example, the step of mixing may comprise vortexing, sonicating and/or rotating, or a combination thereof (e.g., at least two of vortexing, sonicating and rotating, or all three of vortexing, sonicating and rotating).

Vortexing may last, for example, for at least about ten minutes, from about 10 minutes to about 120 minutes, from about 20 minutes to about 60 minutes, or from about 30 minutes to about 45 minutes. Vortexing may be, for example, carried out at a temperature from about 4° C. to about 90° C., from about 10° C. to about 50° C., or from about 18° C. to about 28° C.

Sonicating may last, for example, for at least about 10 minutes, from about 10 minutes to about 60 minutes, from about 20 minutes to about 45 minutes, or for about 30 minutes. Sonicating may be, for example, carried out at a temperature from about 10° C. to about 90° C., from about 20° C. to about 80° C., from about 30° C. to about 70° C., from about 40° C. to about 60° C., or from about 50° C. to about 55° C.

Rotating may last, for example, for at least about one hour, from about 1 hour to about 48 hours, or from about 12 to 24 hours. Rotating may be carried out, for example, at a temperature from about 2° C. to about 25° C., from about 4° C. to about 15° C., or at about 4° C. to about 6° C.

By way of example, the step of mixing may comprise vortexing for 30 to 45 minutes at a temperature from about 18° C. to about 28° C., sonicating for 30 minutes at 55° C., and/or rotating for about 10 hours at 4° C. The step of mixing may comprise vortexing for 30 to 45 minutes at a temperature from about 18° C. to about 28° C. The step of mixing may comprise vortexing for 30 to 45 minutes at a temperature from about 18° C. to about 28° C. and sonicating for 30 minutes at 55° C. The step of mixing may comprise vortexing for 30 to 45 minutes at a temperature from about 18° C. to about 28° C., sonicating for 30 minutes at 55° C. and rotating for about 10 hours at 4° C. It will be appreciated that the step of mixing may be repeated until the solution is substantially transparent. It will be also appreciated that the step of mixing may be influenced by the desired concentration of lipopeptides in the solution.

Merely by way of example, the concentration of lipopeptides in the solution may be from about 0.5 mM to about 2 mM, or from about 1 mM to about 1.75 mM. For example, the concentration of lipopeptides in the solution may be from about 1.25 mM to about 1.55 mM.

A solvent is any liquid substance. The high-ionic strength solvent has an ionic strength that is greater than distilled water. For example the solvent has an ionic strength of at least 20 mM, at least 30 mM, at least 40 mM, at least 50 mM, at least 60 mM, at least 70 mM, at least 80 mM, at least 90 mM, at least 100 mM, at least 110 mM, at least 120 mM, at least 130 mM, at least 140 mM, at least 150 mM, at least 160 mM, at least 170 mM, at least 180 mM, at least 190 mM, at least 200 mM. For example, the solvent has an ionic strength from about 100 mM to about 200 mM. For example, the solvent has an ionic strength from about 125 mM to about 175 mM. For example, the solvent has an ionic strength from about 150 mM to about 170 mM.

The solvent may be serum-free.

The solvent may be selected from the group consisting of cell culture media, phosphate-buffered saline (PBS) or other saline solutions. For example, the cell culture media, phosphate-buffered saline (PBS) and/or saline solution may be serum free. The use of a serum-free cell culture medium may advantageously remove risks associated with contamination, batch-to-batch variability, as well as reduce cell culture costs, and diminish ethical considerations relating to the use of animal sources.

In some examples, in the context of a method of producing a fibrillar supramolecular structure, the solvent may be the same as the aqueous medium in the method for increasing collagen production in a cell and/or method for inhibiting cell migration described herein. Accordingly, the method of producing a fibrillar supramolecular structure and the method of increasing collagen production in a cell and/or for inhibiting cell migration may be combined. Such a combined method may include the step of adding the lipopeptides substantially consisting of ETTES lipopeptides the aqueous medium in the presence of cells.

In a further aspect the present invention provides a solution having an ionic strength that is greater than the ionic strength of distilled water comprising dissolved lipopeptides wherein the lipopeptides substantially consist of ETTES lipopeptides.

It will be appreciated that in this aspect, the dissolved lipopeptides are not self-assembled into a globular supramolecular structure.

In one example, the solution is substantially transparent.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, N Y (1994); and Hale and Marham, The Harper Collins Dictionary of Biology, Harper Perennial, N.Y. (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Aspects of the invention are demonstrated by the following non-limiting examples.

### EXAMPLES

### Materials and Methods

### Preparation of Peptide Amphiphile Solutions

The C₁₆-ETTES PA was solubilised either in deionised water or in DMEM-F12 serum-free medium (SFM) above its critical aggregation concentration (cac) of around 5 mM, and then further diluted to the corresponding final work concentrations (50 and 500 μM). PA solutions were sonicated for 30 min at 55° C. to dissolve the peptide, then transferred under rotation overnight at 4° C. and subsequently stored at 4° C.

### C₁₆-ETTES PA Supramolecular Nanostructure

The PA was characterised using atomic force microscopy (AFM). Briefly, 50 μl aliquots of PA solutions (dissolved either in water or SFM) were drop-spotted onto the surface of borosilicate glass slides and overnight at room temperature inside an aseptic Class II cell culture cabinet. The resulting deposited thin film coatings were washed thrice with deionised water to remove the precipitated salts, dried for another 12 h and then imaged using AFM.

### Cell Culture

Human corneal stromal fibroblasts (hCSFs) were expanded in vitro in serum-containing medium, which was replaced every 2-3 days. Three days prior the cell seeding, hCSFs were serum-starved (cultured in SFM) in order to induce quiescence. Cells were then seeded in 48 well polystyrene tissue culture plates at 3.5×10⁴ cells per cm² in (500 μl of SFM alone or containing C₁₆-ETTES PA at different work concentrations 24 h post-seeding. PA-containing media were replaced every 2 days.

### Biocompatibility and Bioactivity Assays

Biocompatibility and bioactivity of the C₁₆-ETTES PA was monitored for up to 7 days in culture. Cell proliferation was evaluated using the Alamar Blue assay at different time-points and the cell number was calculated by interpolation using a standard curve for the fluorescence values of 1, 5, 10, 20, 50, 100, 150 and 200×10³ cells. Viability assays were also performed at day 7 using live/dead cell staining. Furthermore, the amount of collagen deposited by the cells was investigated using the Sirius Red assay at the end of each experiment. All experiments were performed in triplicate using cells derived from three different donors.

### Cell Migration Assay

The scratch assay was performed to evaluate the effect of the C₁₆-ETTES PA on CSC migration. Briefly, seeded cells were scratched using a 1000 μl tip (producing a scratch of ≈z 1 mm wide), washed twice in PBS to remove the cell debris and then cultured with PA-containing media for 2 days. The closure of the scratch was monitored using the cytonote 6W (Iprasense, France) for live time-lapse images that were taken every 15 min for 48 h. Images were collected and analysed using Image J v1.46. All experiments were performed in triplicate using cells derived from three different donors.

### Statistical Analysis

Error bars represent the standard deviation of the mean. Differences between groups were determined using one- or two-way analysis of variance (ANOVA) with Bonferroni's multiple comparison post hoc tests. Significance between groups was established for p<0.05, 0.01, 0.001 and 0.0001.

### Results

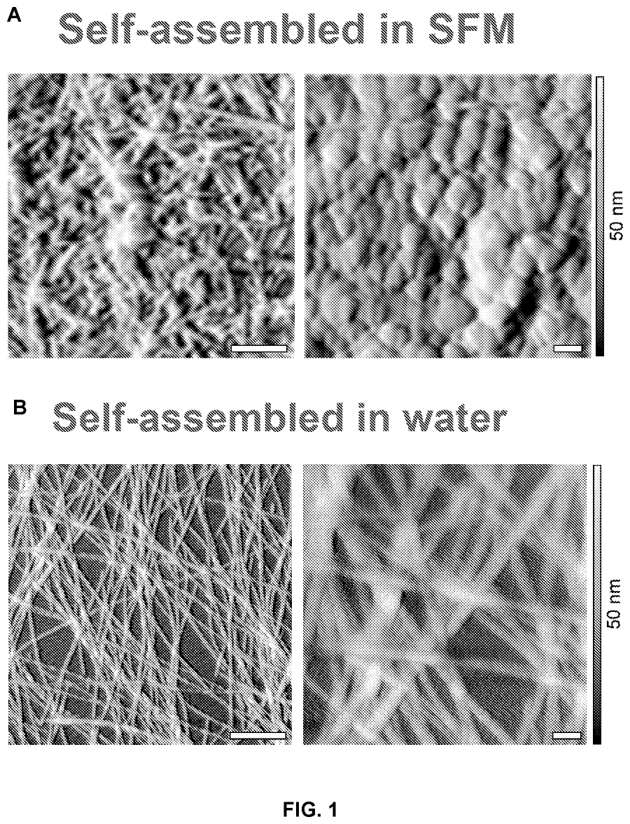

Firstly, the topography of the C₁₆-ETTES PA was studied using atomic force microscopy (AFM) in order to analyse the organisation of the self-assembled nanostructures, and compare its structure when self-assembled in either water or a cationic medium (SFM). The results showed that C₁₆-ETTES self-assembled in medium presented a distinct structure to that observed in water, with much bigger and less defined nanotape networks (FIG. 1). These results confirmed the presence of supramolecular self-assembled structures in medium with a gross difference in structure when compared to water (which in turn indicate a different biological activity and/or function).

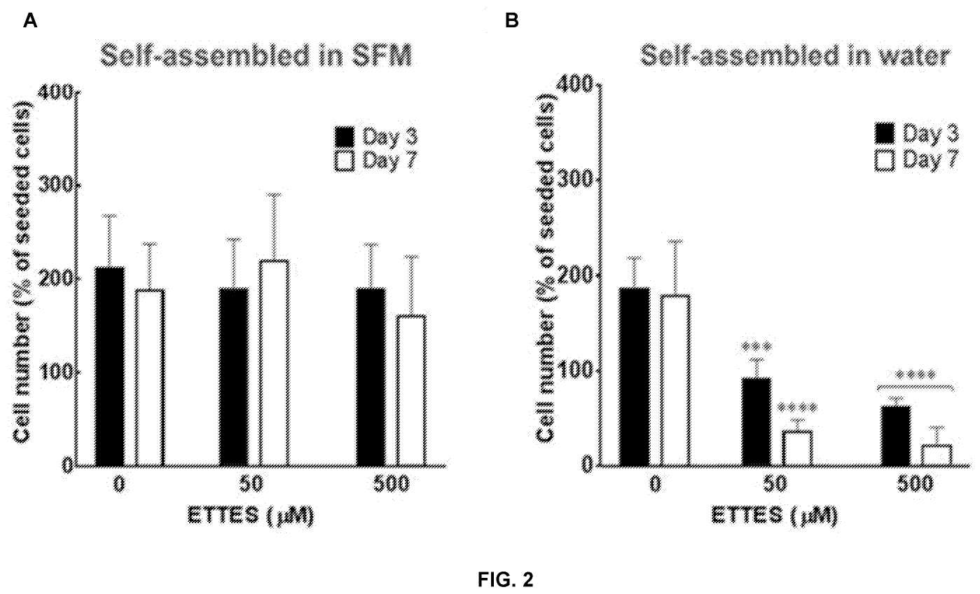

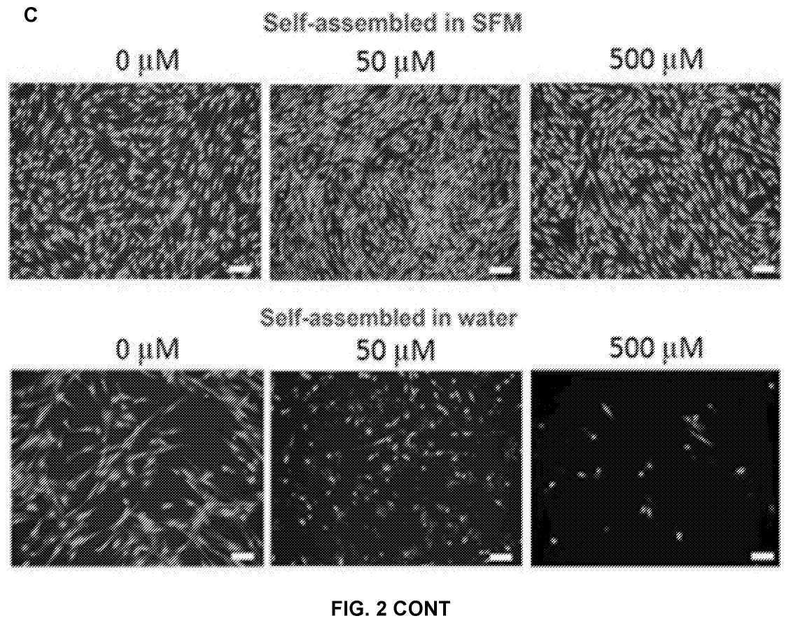

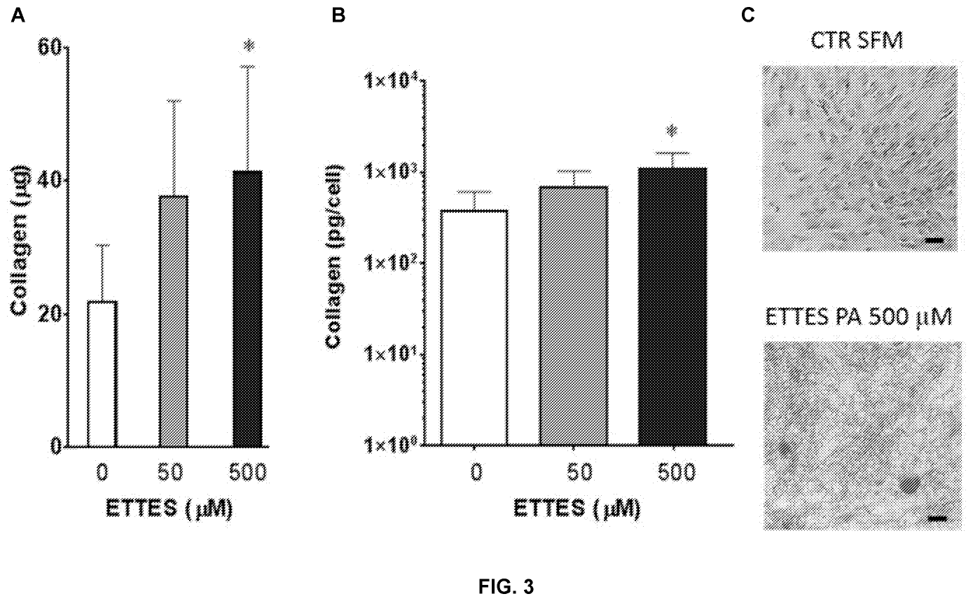

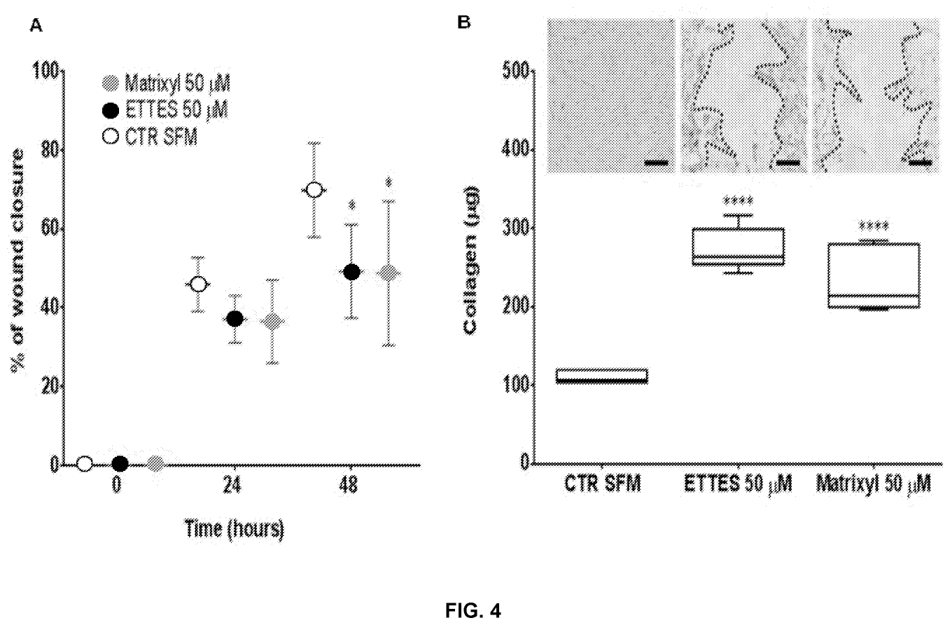

The effect of the PA on cell proliferation, migration, and collagen production was also evaluated. Firstly, the impact of the different PA formulations was tested on hCSF cultures over the course of 7 days. PA molecules solubilised in a cationic solvent (serum-free medium, or SFM) or water, and then added to fresh SFM at 50 and 500 μM and compared with negative controls (0 μM). Results showed that, up to 500 μM, ETTES solubilised in a cationic solvent did not affect cell proliferation (FIG. 2a) whereas equivalent concentrations of PA initially solubilised in water showed to significantly reduce cell number over time (FIG. 2b). These differences were due to the strong cytotoxicity of ETTES solubilised in water, as evidenced by live/dead cell staining assay (FIG. 2c). Specifically, hCSFs cultures with ETTES solubilised in SFM maintained high viability up to 7 days, whereas those treated with PA prepared in water dramatically reduced cell viability (FIG. 2c). Furthermore, ETTES solubilised in SFM at 500 μM significantly increased collagen production, both in bulk (FIG. 3a) and per individual cell (FIGS. 3b and c). Finally, the impact of ETTES on the migration of hCSFs was tested using a cell scratch model. At 50 μM, ETTES significantly reduced cell migration compared to control conditions (FIG. 4a). In addition, collagen production in scratches treated with ETTES was significantly increased (FIG. 4b). Moreover, these effects were comparable with those produced by Matrixyl (FIG. 4). Altogether, results demonstrated the correlation between cell motility and collagen production, thus cells that are secreting/depositing collagen appear to decrease their migration rate. This was observed in a similar study growing cells on RGD coatings (Gouveia et al., 2013).

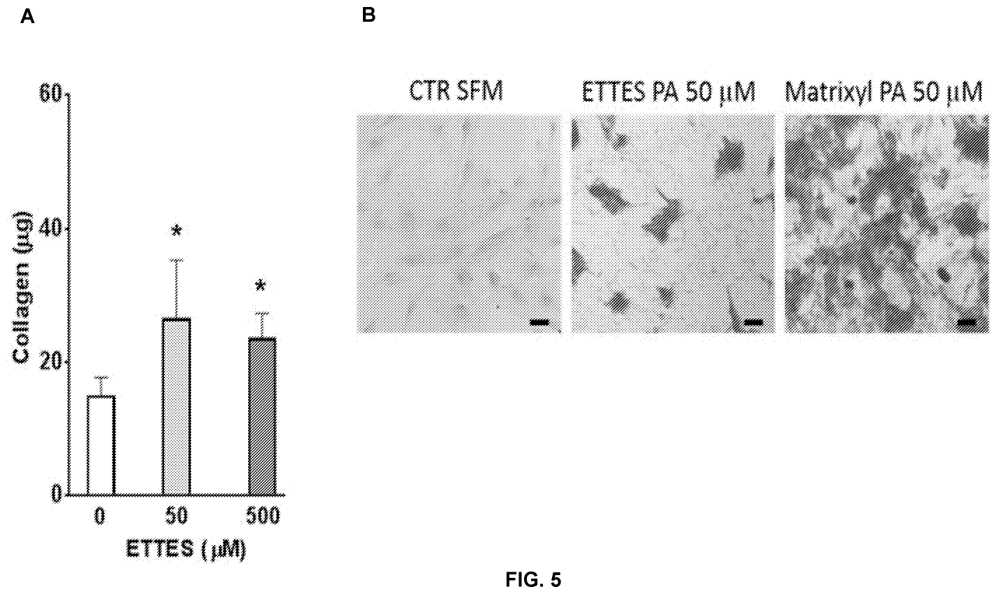

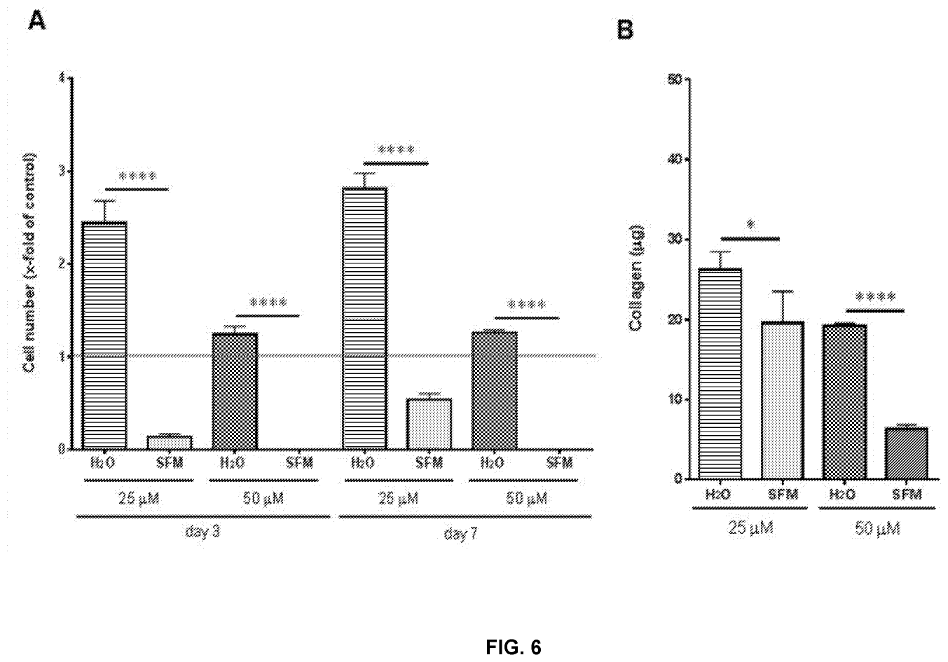

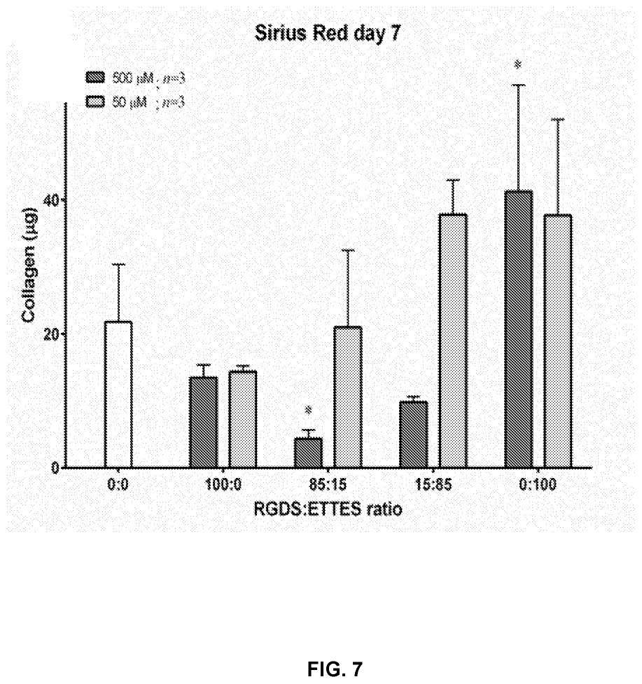

The impact of the different PA formulations was also tested on human adipose-derived mesenchymal stem cells (hASCs) cultures over the course of 7 days. As in previous assays, PA molecules were solubilised in a cationic solvent (SFM) or water, and then added to fresh SFM at 50 and 500 μM and compared with negative controls (0 μM). Results showed that, up to 500 μM, ETTES solubilised in a cationic solvent affected cell viability and proliferation, whereas ETTES solubilised in water maintained it. Furthermore, ETTES solubilised in water up to 500 μM significantly increased collagen production, both in bulk (FIG. 5a) and per individual cell (FIG. 5b). Similar results were obtained when treating C2C12 myoblasts with ETTES, with PA self-assembled in water and diluted in SFM at 25 μM significantly promoting cell proliferation and collagen deposition, whereas, at equivalent concentrations, ETTES initially solubilised in SFM was toxic to cells (FIG. 6).

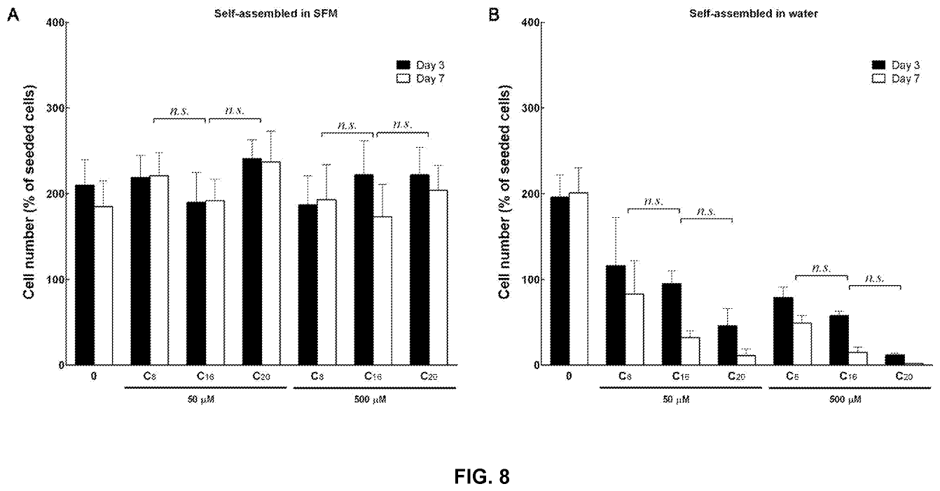

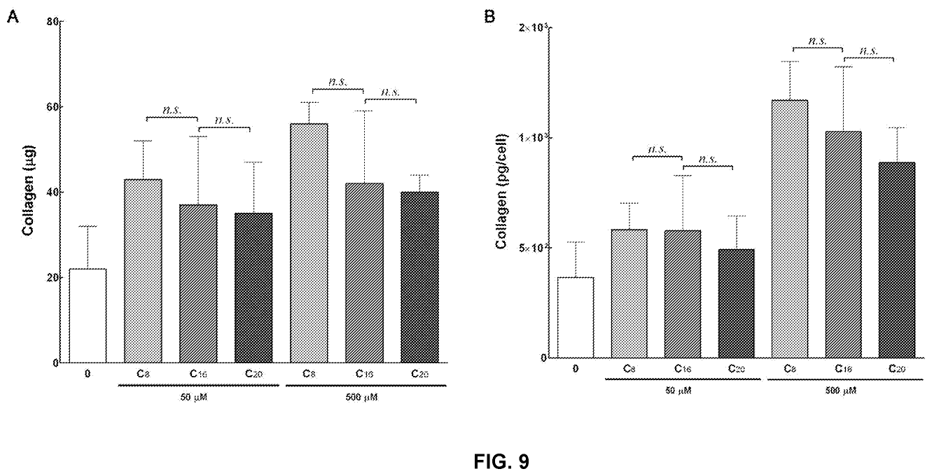

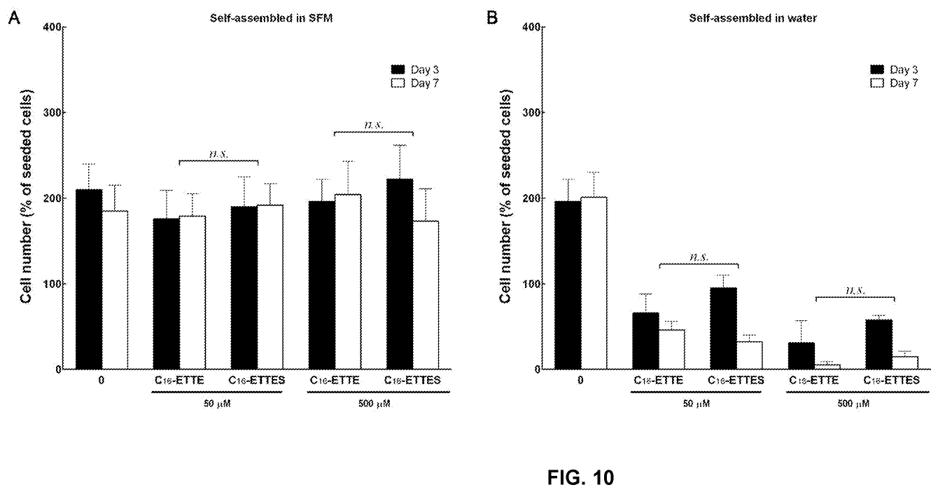

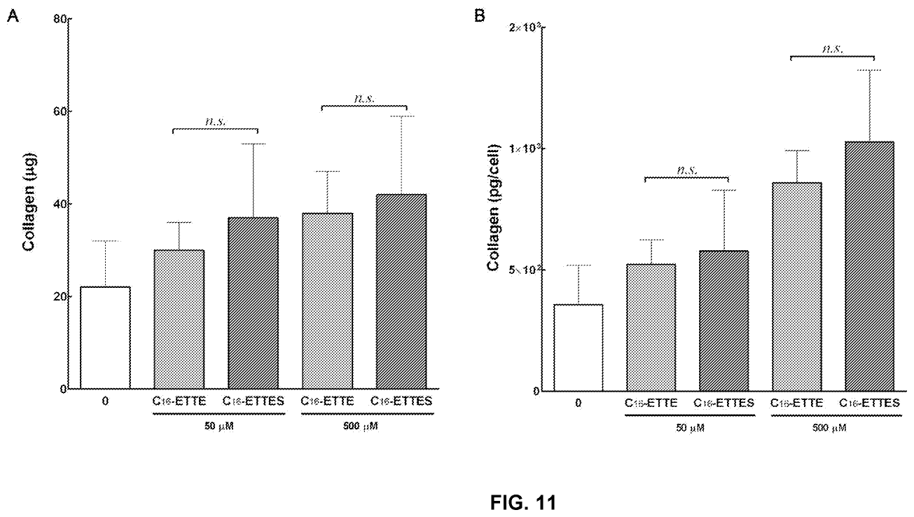

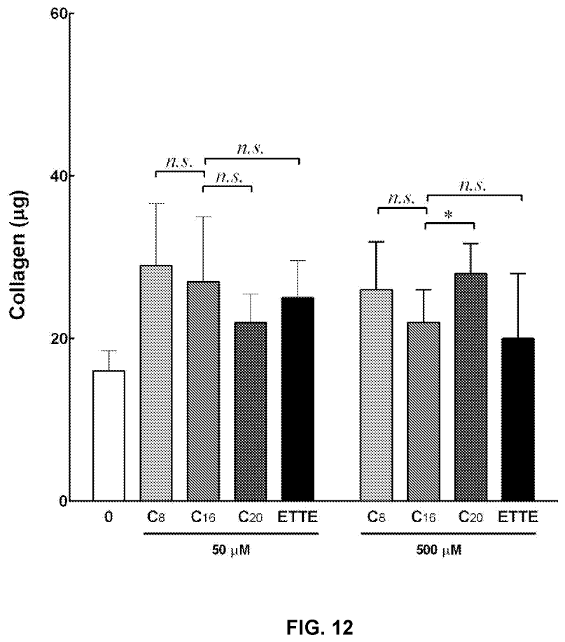

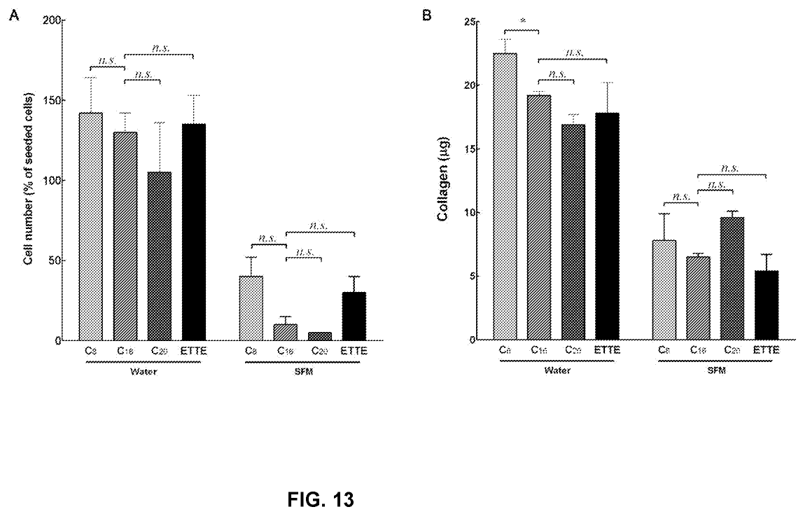

Furthermore, the results presented in FIGS. 8 to 13 confirm that the inventors' findings are not limited to ETTES lipopeptides comprising the full ETTES peptide sequence and a C16 lipid portion. The inventors have also observed an increase in collagen production with C16-ETTES PA variants. For example, as shown in FIG. 9a the inventors have found that, similarly to the C16-ETTES, PAs with C8 and C20 lipopeptide portions similarly result in both bulk and per cell increase of collagen production. Furthermore, assembly of such PAs in water also resulted in reduced cell proliferation as shown in FIG. 8b. Similar results were noted in the context of the C16-ETTES fragment, C16-ETTE.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### SEQUENCES

### SEQ ID NO: 1—ETTES

### REFERENCES

Gouveia, R. M., Castelletto, V., Alcock, S. G., Hamley, I. W., Connon C. J. (2013) Bioactive films produced from self-assembling peptide amphiphiles as versatile substrates for tuning cell adhesion and tissue architecture in serum-free conditions J Mat Chem B, 1, 6157-6169
Gouveia, R. M., Castelletto, V., Hamley, I. W., Connon C. J. (2015) Self-assembling multi-functional templates for the bio-fabrication and controlled self-release of cultured tissue. Tissue Eng Pt A DOI:10.1089/ten.TEA.2014.0671
Castelletto, V., Gouveia R. J., Connon, C. J. Hamley, I. W. (2013) New RGD-Peptide Amphiphile Mixtures Containing a Negatively Charged Diluent. Faraday Discuss, doi:10.1039/C3FD00064H
Jones, R. R., Castelletto, V., Connon, C. J. Hamley, I. W. (2013) Collagen Stimulating Effect of Peptide Amphiphile C16-KTTKS on Human Fibroblasts. Mol. Pharmaceutics, 10 (3), pp 1063-1069.
Hamley I. W. (2011) Self-assembling peptides Soft Matter 7, 4122-4138
Stupp S. I., Zha R. H., Palmer L. C., Cui H., Bitton R. (2013) Self-assembly of biomolecular soft matter Faraday Discuss 166, 9-30

1. A method for increasing collagen production in a cell, the method comprising the step of contacting the cell with lipopeptides, wherein the lipopeptides substantially consist of ETTES lipopeptides, wherein the ETTES lipopeptides comprise an amino acid sequence comprising or consisting of an ETTES (SEQ ID NO: 1) sequence, or a fragment or a variant thereof.

2. A method for inhibiting cell migration, the method comprising the step of contacting the cell with lipopeptides, wherein the lipopeptides substantially consist of ETTES lipopeptides.

3. The method of claim 1 or 2, wherein the cell is a cultured cell.

4. The method of any one of the preceding claims, wherein the cell is cultured in the presence of an aqueous medium comprising suspended therein lipopeptides, wherein the lipopeptides substantially consist of ETTES lipopeptides.

5. The method of any one of the preceding claims, wherein the cell is selected from the group consisting of a stromal cell, a myocyte, a stromal progenitor cell and an adipose derived mesenchymal stem cell, optionally wherein the stromal cell is a corneal stromal cell or a fibroblast.

6. The method of any one of the preceding claims, wherein the cell is an animal cell, preferably a human cell, a monkey cell, a mouse cell, a porcine cell, a bovine cell, or a fish cell.

7. The method of any one of claims 4 to 6, wherein the aqueous medium is selected from the group consisting of cell culture medium, phosphate-buffered saline (PBS) and water.

8. The method of claim 7, wherein the cell culture medium is serum free, and/or DMEM, F-12, or a combination thereof (DMEM-F12).

9. The method of any one of the preceding claims, wherein the ETTES lipopeptides account for at least 90% of the lipopeptides.

10. The method of any one of the preceding claims, wherein the ETTES lipopeptides comprise a lipid portion, wherein the lipid portion comprises or consists of a carbon chain of 6 to 24 carbons.

11. The method of any one of the preceding claims, wherein the ETTES lipopeptides are selected from the group consisting of C₈-ETTES, C₁₆-ETTES, and C₂₀-ETTES lipopeptides.

12. The method of any one of claims 1 to 11, wherein the ETTES lipopeptides are 08-ETTE, C₁₆-ETTE, and C₂₀-ETTE lipopeptides.

13. A pharmaceutical composition comprising lipopeptides, wherein the lipopeptides substantially consist of ETTES lipopeptides, wherein the ETTES lipopeptides comprise an amino acid sequence comprising or consisting of an ETTES (SEQ ID NO: 1) sequence, or a fragment or a variant thereof.

14. The pharmaceutical composition of claim 13, wherein the pharmaceutical composition is in the form of an ointment, gel, cream, liquid, powder, or liniment.

15. The pharmaceutical composition of any one of claim 13 or 14, wherein the pharmaceutical composition is applied onto, absorbed, adsorbed or incorporated into a bandage, a scaffold or sustained-release matrix.

16. The pharmaceutical composition of any one of claims 13 to 15, wherein the ETTES lipopeptides are as defined in any one of claims 9 to 12.

17. A pharmaceutical composition as defined in any one of claims 13 to 16 for use in the treatment of a collagen deficiency disease, for use in enhancing wound healing in a subject, and/or for use in the treatment of cancer.

18. The pharmaceutical composition for use according to claim 17, wherein the collagen deficiency disease is selected from the group consisting of Ehlers-Danlos syndrome, Marfan's syndrome, Osteogenesis imperfecta, brittle bone disease, and collagen vascular disease, optionally wherein the collagen vascular disease is selected from the group consisting of lupus, rheumatoid arthritis, systemic sclerosis, and temporal arteritis.

19. Use of a pharmaceutical composition, wherein the pharmaceutical composition is as defined in any one of claims 13 to 16, wherein the use is non-therapeutic.

20. The use according to claim 19, wherein the non-therapeutic use is to improve or restore a subject's appearance.

21. The use according to claim 20, wherein the subject's appearance is improved or restored by reducing or preventing skin wrinkles, reducing or preventing skin hyperpigmentation, and/or increasing skin elasticity or preventing loss of skin elasticity.

22. A fibrillar supramolecular structure comprising lipopeptides, wherein the lipopeptides substantially consist of ETTES lipopeptides, wherein the ETTES lipopeptides comprise an amino acid sequence comprising or consisting of an ETTES (SEQ ID NO: 1) sequence, or a fragment or a variant thereof.

23. A method of producing a fibrillar supramolecular structure comprising lipopeptides, wherein the lipopeptides substantially consist of ETTES lipopeptides, wherein the ETTES lipopeptides comprise an amino acid sequence comprising or consisting of an ETTES (SEQ ID NO: 1) sequence, or a fragment or a variant thereof, the method comprising dissolving the lipopeptides in a solvent having an ionic strength that is greater than the ionic strength of distilled water to produce the supramolecular structure.

24. The fibrillar supramolecular structure of claim 22 or the method of claim 23, wherein the ETTES lipopeptides are as defined in any one of claims 9 to 12.

25. The method of any one of claim 23 or 24, wherein the solvent has an ionic strength of at least 100 mM.

26. The method of any one of claims 23 to 25, wherein the lipopeptides are lyophilised prior to dissolving.

27. The method of any one of claims 23 to 26, wherein the dissolving comprises a step of mixing the lipopeptides in the solvent to obtain a substantially transparent solution.

28. The method of any one of claims 23 to 27, wherein the solvent is cell culture medium.

29. The method of any one of claims 23 to 29, wherein the cell culture medium is serum free, and/or DMEM, F-12, or a combination thereof (DMEM-F12).

30. Use of lipopeptides comprising an amino acid sequence comprising or consisting of an ETTES (SEQ ID NO: 1) sequence, or a fragment or a variant thereof, in a method for producing tissue in vitro, optionally wherein the tissue is meat.
